(19) United States
(12) Patent Application Publication
(10) Pub. No.: US 2005/0048108 A1
(43) Pub. Date: Mar. 3, 2005

(54) Sulphur-containing amphiphilic agents for the transfer of biologically active molecules into cells

(75) Inventor: Oliver Keil, Schildow (DE)

Correspondence Address: BIRCH STEWART KOLASCH & BIRCH, PO BOX 747, FALLS CHURCH, VA, 22040-0747, US

(73) Assignee: G.O.T. Therapeutics GMBH, 13125 Berlin (DE)

(21) Appl. No.: 10/451,687

(22) PCT Filed: Dec. 21, 2001
(86) PCT No.: PCT/DE01/04843

(30) Foreign Application Priority Data
Dec. 27, 2000 (DE) 100 64 870.3

(51) Int. Cl. A61K 9/127; C07C 317/24

(52) U.S. Cl. 424/450; 554/85

(57) ABSTRACT

The present invention relates to amphiphilic, cationic sulpho-substituted phosphatidyl ethanolamine analogs and their salts, which are capable of complexing biopolymers such as DNA, RNA, oligonucleotides, ribozymes, proteins and peptides and to infiltrate them into eukaryotic cells. Particularly suitable are compounds which are derived from 1,2-dioleoyl-3-sn-phosphatidyl ethanolamine (DOPE) and in which the phosphoric acid ester group of DOPE is replaced by an isosteric group CH₂—SO—CH₂ or CH₂—S(O)₂—CH₂. Because of their property of forming aggregates with biologically active molecules, such as for example DNA or RNA, there compounds are particularly suitable for application in gene therapy, but also for diagnostic purposes.

### SPECIFICATION

The present invention relates to cationic sulpho-substituted phosphatidyl ethanolamine analogs and their salts, which are capable of infiltrating biologically active macromolecules (particularly DNA and RNA) into eukaryotic cells.

In the last ten years, the transfer of biopolymers, particularly of DNA, RNA and oligonucleotides, into eukaryotic cells has developed into a fundamental field of work in molecular biology and molecular medicine (P. A. Martin, S. M. Thomas; Human Gene Therapy 9 (1998) 87-114). Of particular interest here are above all gene therapeutic approaches, but also diagnostic medicine. Methods partially established heretofore for the insertion of DNA into eukaryotic cells depend on infiltrating the DNA sequence concerned with the aid of replication deficient, recombinant retro-, adeno-, or adeno-associated viruses. However, these have the disadvantage that until now they have been nearly exclusively restricted to ex vivo applications, since the viral proteins sometimes lead to intense immune reactions, and replication-competent viruses cannot always be excluded. Retroviruses have the further disadvantage that they integrate unspecifically but firmly into the host genome and thus can potentially result in malignant mutations. Because of these properties, it is understandable that these methods set extremely high safety requirements and can only be carried out with great financial expense. Biophysical methods, such as, for example, the bombardment of cells with DNA-laden gold particles (“biolistics”), or electroporation, are likewise only usable ex vivo for obvious reasons. The long known calcium phosphate coprecipitation and the DEAE-dextran methods appear little suitable because of their low efficiency .

A further method, developed in recent years, for the infiltration of biologically active macromolecules into eukaryotic cells uses cationic polymers such as polylysine, polyethylenimine, or PAMAM-dendrimeres. Polyanions such as DNA, RNA or oligonucleotides form aggregates with these by electrostatic mutual action and in this form are taken up by the cells, presumably by endocytosis.

However, poly-L-lysine has to be modified beforehand by chemical reaction with receptor ligands (e.g., transferrin, glycoproteins) (E. Wagner et al., Proc. Natl. Acad. Sci. USA 87 (1990) 3410-3414) or endosmolytic peptides (E. Wagner, Proc. Natl. Acad. Sci. USA 89 (1992) 7934-7938) in order to have sufficient efficiency. Therefore, because of their polymeric nature, compounds of this type are very heterogeneously composed mixtures of substances and can be produced in a defined form only at great expense.

A further method, which has assumed great importance in recent years, is based on the ground-breaking work of Feigner et al. Here liposomal or even micellar structures are generated from cationic, lipid amphiphiles, pure or mixed with neutral phospholipids such as dioleoylphosphatidyl ethanolamine (DOPE) (Feigner et al., WO 91/16024). Due to electrostatic mutual action with anionic biopolymers (such as DNA or RNA), these form aggregates which are thereafter efficiently taken up by eukaryotic cells. DNA can thus be transported into the cell nucleus and then leads to the expression of the corresponding protein. Although the mechanism for this has until now not been elucidated, there is however agreement that the aggregates reach the cell interior by endocytotic processes by means of endosomes. So that the internalized biopolymers can reach other cell compartments (e.g., cytoplasm or cell nucleus), they must emerge from the endosomes, since they would otherwise be enzymatically decomposed in the lysosomes. In most cases, this is attained by the admixture of the phospholipid DOPE, which because of its particular conical geometry is capable of inducing inverted hexagonal liquid crystalline phases well below (about 10° C.) a physiological temperature of 37° C. (J. O. Rädler et al., Science 281 (1998) 78-81). These have a high tendency to fuse with double-layer structures (e.g., biological membranes, endosomal membranes). In an article by Szoka et al., it is postulated that by this process of fusion, anionic, cellular lipids neutralize the charge of the infiltrated cationic lipids and thus release the complexed DNA into the cytosol (Szoka et al., Biochemistry 35 (1996) 5616-5623).

Since the first description by Felgner, numerous cationic amphiphiles, mostly empirically found, have been synthesized for the transfer of anionic macromolecules, such as e.g. DNA (A. D. Miller, Angew. Chem. 110 (1998) 1862-1880; L. Huang, X. Gao, Gene Therapy 2 (1995) 710-722).

The cationic amphiphiles known heretofore have the disadvantage that they cannot induce inverted hexagonal phases. They therefore have hardly any fusogenic properties, and auxiliary lipids such as, e.g., DOPE, have to be admixed. A few lipids, such as, for example, DOGS (J. P. Behr et al., Proc. Natl. Acad. Sci. 86 (1989) 6982-6986), are not capable of forming double layered structures and are present in micellar structures. Here also auxiliary lipids have to be admixed in order to attain a high gene transfer efficiency. Most reagents described heretofore are therefore multi-component mixtures, and the auxiliary lipids used, e.g., DOPE, are sensitive to hydrolysis and oxidation. The production of the biologically active formulations is therefore connected with considerable expense from the pharmaceutical standpoint, since the quality of each individual component has to be ensured. Many of the known cationic lipids, such as, e.g., DOTMA or DELRIE, have the disadvantage that because of ether bonds they are metabolized only with difficulty and therefore show distinct cell-toxic properties.

The invention thus has as its object to make available new amphiphiles for the transfer of biopolymers (particularly of DNA and RNA) into eukaryotic cells, which
are easily metabolized and show little cell toxicity,
are capable of inducing hexagonal phases and thus possess strongly fusogenic properties, and at the same time are cationically charged,
can be produced easily and cost-favorably in large quantities,
and which require as small as possible an admixture of (helper) lipids.

A transfer of biomolecules, particularly of DNA and RNA, into eukaryotic cells (transfection) which is surprisingly efficient in regard to the said requirements can be attained by the use according to the invention of sulfur-containing amphiphiles of the general formula I, 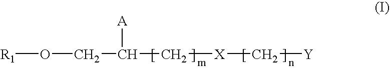
wherein
R₁ denotes a straight or branched chain, saturated or unsaturated alkyl or acyl residue with 10-24 carbon atoms,
A denotes a group O—R₂ or CH₂—O—R₂, in which R₂ has the meaning given for R₁ and may be the same as R₂ or different, where with the presence of a steric center, the methine carbon atom connected to A can be present in R- or else S-configuration or racemic,
X denotes a group 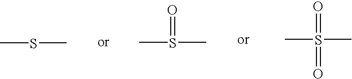
Y denotes a group N⁺R₃R₄R₅Z⁻ or a group NR₃R₄, where R₃-R₅, independently of each other, denote hydrogen, an alkyl group with 1-4 carbon atoms, a group —(CH₂)ᵢ—OH, or a group —(CH₂)ᵢ—NH₂ with i=2-6, and Z⁻ denotes a pharmaceutically acceptable anion, and where m and n, independently of each other, denote an integer 1-6.

Preferred here are such compounds in which the residue R₁ is an alkyl or acyl residue from the group of laur(o)yl, myrist(o)yl, palmit(o)yl, stear(o)yl, ole(o)yl, lin(o)yl, and linole(o)yl, and m=2 and n=3. Most particularly suitable are molecules in which R₁ is a lauroyl, myristoyl or oleoyl residue, the group A is a lauroyloxy, myristoyloxy or oleoyloxy residue, m=2 and n=3, X is a —SO— or —SO₂— group, and Y is an —NH₃⁺Z⁻ group or —NH₂. In a preferred embodiment, the pharmaceutically acceptable anion is an ion from group halide, acetate, trifluoroacetate, mesylate, besylate, phosphate, tartrate, or citrate.

The lipids according to the invention are suitable for the transfer of biologically active macromolecules, particularly DNA and RNA, into eukaryotic cells. The lipids can be present in aqueous (liposomal) dispersion or as a solution in water, and at the same time other, already known lipids, such as phospholipids or membrane-associated steroids, can be admixed. The DNA (RNA) can be pre-complexed with polycations, particularly with protamine sulfate. In contrast to the transfection agents known heretofore, the compounds according to the invention have a series of decisive advantages. Molecules of the described kind are used which are structurally very close to phosphatidyl ethanola-mine lipids.

The structural unit 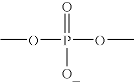
of the phosphatidyl ethanolamine is replaced in the lipids according to the invention by the isosteric groups 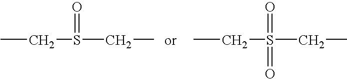
Because of the structural analogy, they potentially have a conical molecular geometry and therefore possess strong membrane fusogenic properties. However, the negative charge of the phosphoric acid ester unit is avoided, so that the lipids according to the invention are positively charged. They can therefore enter into electrostatic mutual action with negatively charged biomolecules and complex these. A further advantage is that the sulfo-analog lipids, like other sulfoxides or sulfones (e.g., DMSO), possess a high ability to pass into membranes. In contrast to many heretofore known cationic lipids, the lipids according to the invention furthermore have ester bonds and are therefore easily metabolized and hence potentially non-toxic.

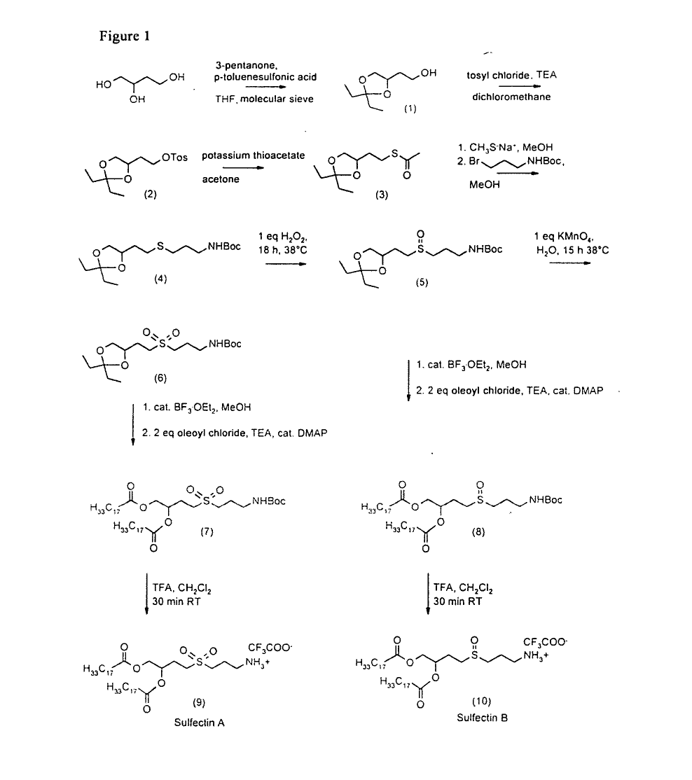

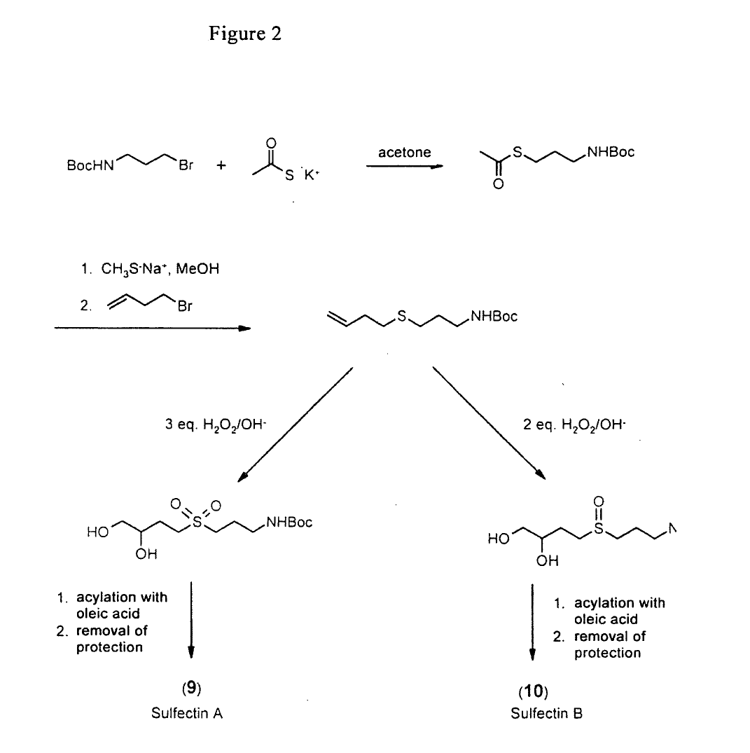

The production of the compounds takes place from inexpensive starting chemicals using methods familiar to one skilled in the art (Methods of Organic Chemistry (Houben-Weyl), 4th. edition, Thieme Verlag (Stuttgart) 1952) and protective group techniques (T. Greene, P. Wuts; Protective Groups in Organic Synthesis, 2d. ed., John Wiley & Sons (New York) 1991). The course of the reaction is shown in FIG. 1. Alternatively to this, the lipids according to the invention can also be synthesized according to the course of the reaction described in FIG. 2.

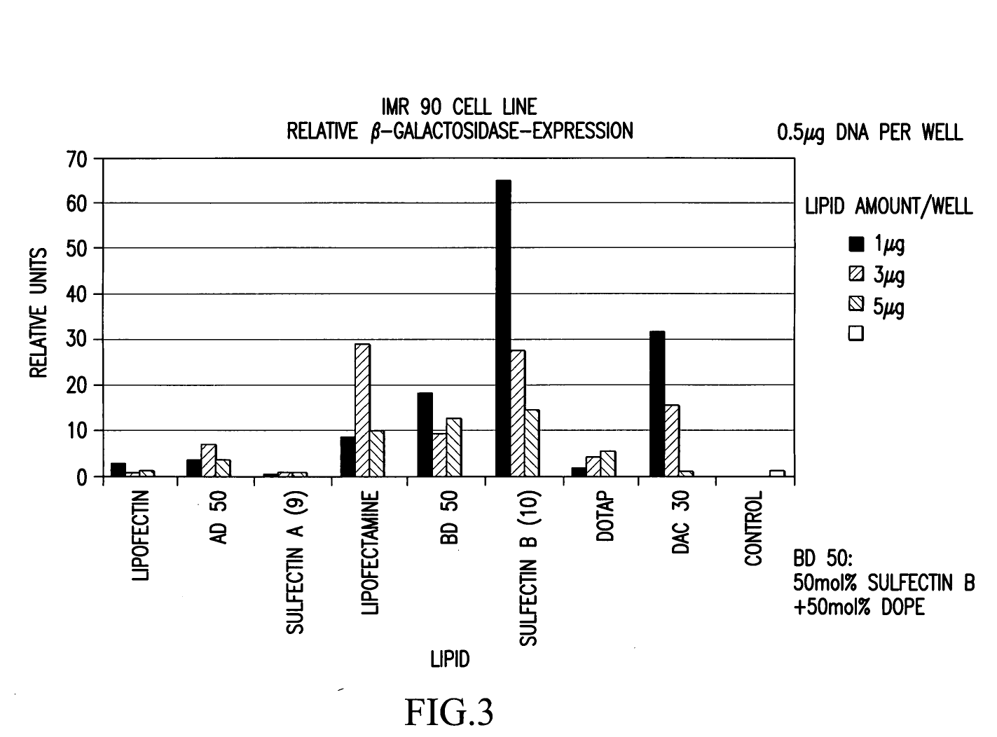

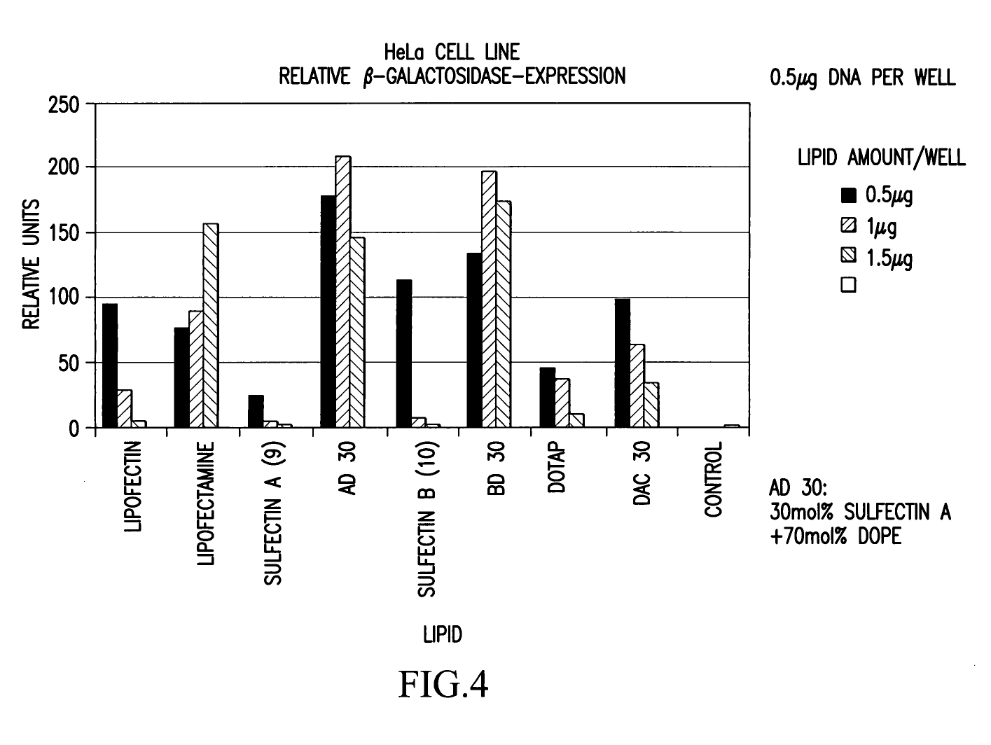

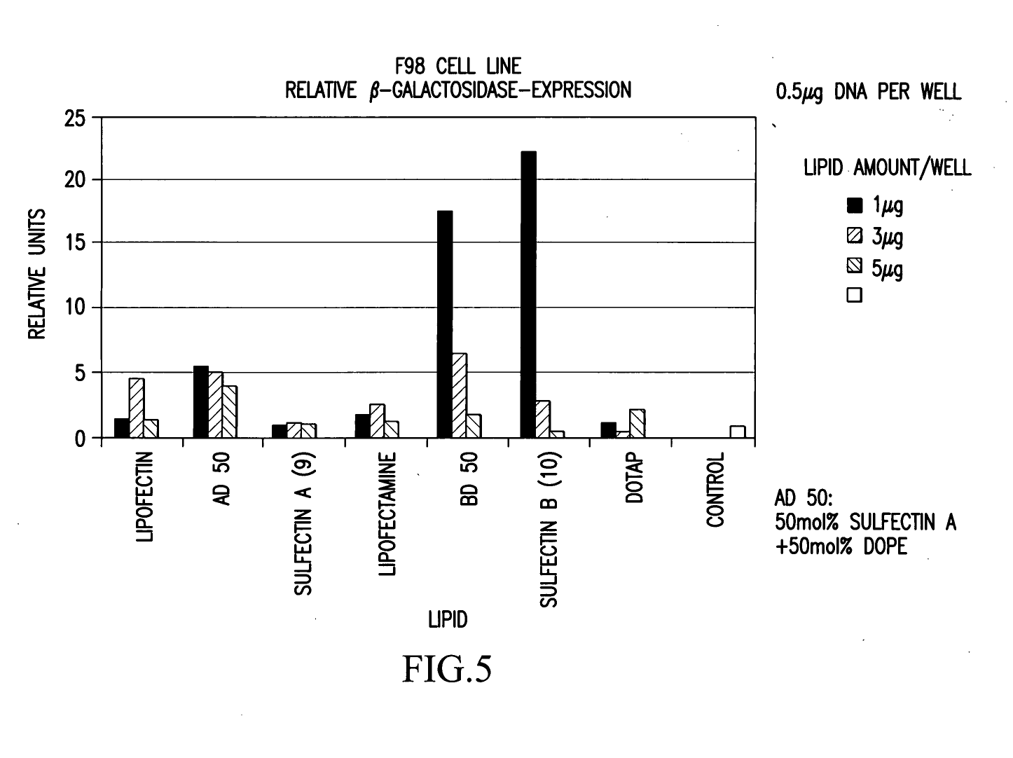

The gene transfer properties of the lipids according to the invention (lipid 9 (Sulfectin A) and lipid 10 (Sulfectin B)) were tested on various cell lines and were compared with the reagents known heretofore (FIGS. 3, 4 and 5). A plasmid coded for β-galactosidase was used as the reporter system. In the trials, the lipids were also mixed with, among other things, the neutral auxiliary lipid 1,2-dioleoyl-sn-glycero-3-phosphoethanolamine (DOPE). With the lipids 9 and 10 it is possible to transfect all investigated cell lines with markedly higher efficiency than with the known lipids Lipofectin®, LipofectAMINE™ (Life Technologies, USA), DOTAP (Roche Diagnostics, Germany), and DAC-30 (Eurogentec, Belgium). The gene transfer efficiency of the lipids is compared and shown in FIGS. 3-5. In comparison with the commercial lipids, the lipids according to the invention make possible a surprisingly high gene expression. Among others a β-galactosidase expression up to 10 times higher was found. This is the more surprising in that in most cases (IMR90 and F98 cells) no admixture of an a helper lipid was necessary. With all the commercial lipids, on the other hand, a portion of DOPE was indispensable for an efficient gene transfer. The high gene transfer efficiency of the pure lipid is also advantageous for the production of the gene transfer reagent, since the formulation step of the cationic lipid and auxiliary lipid is omitted. Problems which are attributable to the oxidation sensitivity of DOPE cannot arise. Furthermore the possibility does not exist that the gene transfer properties are negatively affected by lyso-DOPE (hydrolysis product).

Although the reaction paths and reagents used, shown in the following examples and Figures, represent preferred embodiments, the scope of the invention is not to be limited thereby.

### EXAMPLE 1

### Preparation of 2-(2,2-diethyl-1,3-dioxolan-4-yl)ethanol (1)

In a 250-ml round flask, 10.61 g (0.1 mol) of 1,2,4-butanetriol are dissolved in 70 ml anhydrous THF [tetrahydrofuran] and treated with 16 ml (0.15 mol) 3-pentanone and 580 mg toluenesulfonic acid. Thereupon 19 g of freshly dried molecular sieve are added and the mixture is well shaken for 48 hours at room temperature.

After this time, 5 g of Ambersep 900 OHΓ ion exchanger are added, shaken for 20 min, then filtered. The filtrate is freed from solvent on a rotary evaporator and the residue is taken up in 170 ml ethyl acetate. The solution is washed three times, each time with 15 ml saturated NaHCO₃ solution, and three times, each time with 15 ml saturated NaCI solution. The organic phase is dried over Na₂SO₄, filtered, and freed from solvent on the rotary evaporator. After drying in vacuum, 5.90 g (34 mmol) of a viscous, colorless oil are obtained.

### EXAMPLE 2

### Preparation of 2-(2,2-diethyl-1,3-dioxolan-4-yl)ethyl-1-tosylate (2)

In a 250-ml round flask, 5.90 g (34 mmol) of compound 1 are dissolved in 50 ml anhydrous dichloromethane and treated with 9.5 ml of triethylamine. Then 7.15 g (37.5 mmol) of tosyl chloride dissolved in 100 ml of anhydrous dichloromethane are dropped in via a dropping funnel during 20 min with stirring at room temperature. The mixture is stirred for 24 h at room temperature and then treated with 557 μl (5,1 mmol) of N,N-dimethylethylenediamine (in order to destroy excess tosyl chloride). The solution is transferred to a separating funnel and then washed with water and dilute sodium chloride. Finally it is again washed once with 15 ml saturated NaHCO₃ solution and once with 15 ml saturated NaCl solution. The organic phase is dried over Na₂SO₄, filtered, and freed from solvent on the rotary evaporator. After drying in vacuum, 10.21 g (31.2 mmol) of a viscous, very light yellow oil are obtained.

### EXAMPLE 3

### Preparation of S-acetyl-2-(2,2-diethyl-1,3-dioxolan-4-yl)ethyl-1-thiol (3)

In a 250 ml round flask, 6.0 g (18.3 mmol) of compound 2 are dissolved in 150 ml acetone and treated with 4.18 g (36.6 mmol) finely powdered potassium thioacetate. The mixture is stirred under argon for 72 h at room temperature and then filtered from the solid. The filtrate is freed from solvent on the rotary evaporator and the residue is taken up in 170 ml ethyl acetate.

The solution is washed in a separating funnel six times, each with 40 ml of water, and then once with 40 ml of saturated NaCl solution. The organic phase is dried over Na₂SO₄, filtered, and freed from solvent on the rotary evaporator. After drying in vacuum, 4.10 g (17.6 mmol) of a reddish brown oil are obtained.

### EXAMPLE 4

### Preparation of 3-{[2-(2,2-diethyl-1,3-dioxolan-4-yl)ethyl]thio}-propyl-1-tert-butylcarbamate (4)

In a 25 ml side connection flask, 1.243 g (5.35 mmol) of compound 3 are placed under argon and are treated with 5.35 ml of a 1 M solution of sodium methanethiolate in anhydrous methanol. The mixture is stirred at room temperature for 30 min and the solvent is then removed in vacuum with slight heating. The remaining red-brown paste is further dried for 15 min in vacuum; the flask is then flushed with argon and the mass is dissolved in 5 ml anhydrous methanol. Thereupon a solution of 1.062 g (4.46 mmol) 3-(boc-amino)propyl bromide are added in 5 ml anhydrous methanol, and the mixture is stirred under argon for 4 hours at room temperature. The solvent is removed on the rotary evaporator and the residue is taken up in 150 ml ethyl acetate. The solution is washed four times each with 20 ml water and twice each with 15 ml saturated NaCl solution. The organic phase is dried over Na₂SO₄, filtered, and freed from solvent on the rotary evaporator. After drying in vacuum, 2.04 g of a reddish brown crude product are obtained. Subsequent chromatographic purification on 50 g silica gel 60 with hexane/acetic ester as eluent gave 1.55 g (4.46 mmol) of a colorless oil.

### EXAMPLE 5

### Preparation of 3-{[2-(2,2-dietbyl-1,3-dioxolan-4-yl)ethyl]-sulfinyl}-propyl-1-tert-butylcarbamate (5)

In a 50 ml round flask with an attached gas discharge tube, 648 mg of compound 4 are placed and treated with 169 μl of a 35% hydrogen peroxide solution. The mixture is heated for 15 h at 38° C. with strong stirring, after which a homogeneous phase forms. After this time, the residue is taken up in 50 ml of ethyl acetate and stirred with a little anhydrous Na₂SO₄. It is then filtered off, thoroughly washed with ethyl acetate, and the solvent is removed on the rotary evaporator. The subsequent chromatographic purification on 50 g silica gel 60 with dichloromethane/methanol (13:1) as eluent gave 594 mg (1.63 mmol) of a colorless, viscous oil.

### EXAMPLE 6

### Preparation of 3-{[2-(2,2-diethyl-1,3-dioxolan-4-yl)ethyl]-sulfonyl}-propyl-1-tert-butylcarbamate (6)

In a 50 ml round flask with an attached gas discharge tube, 352 mg of compound 5 are placed and treated with a solution of 158 mg of potassium permanganate in 2 ml of saturated NaHCO₃ solution. The mixture is heated to 35-38° C. with stirring for 15 h. The residue is thereafter intensively stirred with 70 ml of ethyl acetate and then filtered off from the insoluble manganese dioxide. The organic phase is dried over Na₂SO₄, filtered, and freed from solvent on the rotary evaporator. The subsequent chromatographic purification of the crude product on 50 g silica gel 60 with dichloromethane/methanol (20:1) as eluent gave 301 mg (0.793 mmol) of a colorless, very viscous oil.

### EXAMPLE 7

### Preparation of 3-[(3,4-dioleoyloxybutyl)-sulfonyl]propyl-1-tert-butylcarbamate (7)

In a 10 ml side connection flask, 174 mg (459 μmol) of compound 6 are dissolved under argon in 1.5 ml anhydrous methanol. Over a period of 8 hours, six portions each of 100 μl of a solution of 23.2 μl BF3 etherate in 2 ml anhydrous MeOH are added with stirring at room temperature. Thereafter 100 mg of a dry Ambersep 900OHΓ ion exchanger are added and stirred for 5 min. After filtering, the solvent is removed on the rotary evaporator and the residue is purified by chromatography on 20 ml silica gel 60 with dichloromethane/methanol (10:1) as eluent. 81 mg (260 μmol) of a colorless, very viscous oil are obtained.

This is dissolved in 3.5 ml of anhydrous dichloromethane in a 10 ml found flask and treated with 111 μl of triethylamine. With stirring and cooling, 210 mg of oleoyl chloride (purity (GC) 99%) is slowly added via a syringe. Further, a catalytic amount of DMAP is added, and the mixture is stirred for 18 h at room temperature in a darkened flask. The solution is then transferred with 40 ml dichloromethane into a separating funnel, and is washed twice, each time with 5 ml of water slightly acidified with HCl, and once with 5 ml of water. It is then washed once again with water made slightly alkaline (NaHCO₃), and the organic phase after drying over Na₂SO₄ is freed from solvent on the rotary evaporator. Subsequent chromatographic purification on 20 g silica gel 60 with hexane/ethyl acetate (2:1) as eluent gave 105 mg (125 μmol) of a colorless, viscous oil.

### EXAMPLE 8

### Preparation of 3-[(3,4-dioleoyloxybutyl)-sulfinyl]propyl-1-tert-butylcarbamate (8)

In a 10 ml side connection flask, 200 mg (550 μmol) of compound 5 are dissolved under argon in 1.5 ml of anhydrous methanol.

Then over a period of 8 hours, six portions each of 100 μl of a solution of 27.6 μl BF₃ etherate in 2 ml anhydrous MeOH are added with stirring at room temperature. After this, 120 mg of a dry Ambersep 900 OHΓ ion exchanger are added and stirred for 5 min. After filtering off, the solvent is removed on the rotary evaporator and the residue (182 mg) is purified by chromatography on 20 g silica gel 60 with dichloromethane/methanol (9:1) as eluent. 66 mg (223 μmol) of a colorless, very viscous oil are obtained.

In a round flask, this is dissolved in 3.5 ml of anhydrous dichloromethane and treated with 125 μl of triethylamine. With stirring and cooling, 250 mg of oleoyl chloride (purity (GC) 99%) are slowly added via a syringe. A further catalytic amount of DMAP is added and the mixture is stirred for 18 h at room temperature in the darkened flask. With 40 ml of dichloromethane, the solution is then transferred into a separating funnel and washed twice, each time with 5 ml of slightly HCl-acidified water, and once with 5 ml of water. Then after washing once again, with 5 ml slightly alkaline water (NaHCO₃), the organic phase after drying over Na₂SO₄ is freed from solvent on the rotary evaporator. Subsequent chromatographic purification of the crude product on 20 g silica gel 60 with hexane/acetic ester (1:1) as eluent gave 64 mg (78 μmol) of a colorless, viscous oil.

### EXAMPLE 9

### Preparation of 3-[(3,4-dioleoyloxybutyl)-sulfonyl]propyl-1-ammonium trifluoroacetate (9) (Sulfectin A)

In a 10 ml side-connection flask, under argon, are placed 52 mg (62 μmol) of compound 7, dissolved in 600 μl of anhydrous dichloromethane. With cooling and stirring, 400 μl of trifluoroacetic acid are then added and the mixture is stirred for 30 min at room temperature. The solvent is then removed in high vacuum without supplying heat, and the residue is dried for 1 h in vacuum. The remaining lipid film is taken up twice with 1.5 ml anhydrous dichloromethane each time, and is freed from solvent in vacuum. In conclusion, this procedure is repeated once with 1 ml of methanol and the lipid film is finally dried for 3 h in high vacuum. 52.4 mg (61.3 μmol) of a colorless lipid film are obtained.

### EXAMPLE 10

### Preparation of 3-[(3,4-dioleoyloxybutyl)-sulfinyl]propyl-1-ammonium trifluoroacetate (10) (Sulfectin B)

In a 10 ml side-connection flask, under argon, are placed 44 mg (54 μmol) of compound 8, dissolved in 600 μl of anhydrous dichloromethane. With cooling and stirring, 400 μl of trifluoroacetic acid are then added and the mixture is stirred for 30 min at room temperature. The solvent is then removed in high vacuum without supplying heat, and the residue is dried for 1 h in vacuum. The remaining lipid film is taken up twice with 1.5 ml anhydrous dichloromethane each time, and freed from solvent in vacuum. In conclusion, this procedure is repeated once with 1 ml methanol and the lipid film is finally dried for 3 h in high vacuum, and 45 mg (53.6 μmol) of a colorless lipid film are obtained.

### EXAMPLE 11

### Liposomal Formulation of the Lipids According to the Invention for the Transfection Experiments

Lipids 9 and 10 are dissolved in chloroform and dried to a lipid film in vacuum. If necessary, they are previously mixed with 1,2-dioleoyl-sn-glycero-3-phosphoethanolamine (DOPE) in different mol percent ratios. The remaining solvent residues are removed in high vacuum. The lipid films are then rehydrated in sterile water, and the liposomes are generated by ultrasonic treatment. The total lipid concentration of the resulting dispersion is 1 mg/ml.

### EXAMPLE 12

### Transfection of Adherent Cell Lines

General: The cell lines used, HeLa (human cervical carcinoma), F98 (rat glioblastoma), IMR90 (human embryonic lung) are cultivated under standard conditions (according to ATCC or EACC data) at 37° C./5% CO2 (HeLa/MEM-medium, F98/DMEM medium, IMR90/DMEM medium). The commercial transfection reagents Lipofect-AMINE™, Lipofectin®, DOTAP and DAC-30 were used according to the manufacturer's data.

### EXAMPLE 13

### Transfection with pUT 651 Plasmid

pUT 651 plasmid (CAYLA, France) was used as the reporter gene; it codes for lacZ under control of a CMV promoter.

On the day before transfection, 5,000-10,000 cells per well were seeded on a 96-well culture plate, so that for transfection they had a confluence of about 70%. The medium was replaced by 80 μl/well of fresh medium (10% FCS) shortly before the transfection.

The lipoplexes are prepared on a separate 96-well plate, in that 2 μg of DNA in 40 μl serum-free medium is mixed with 2-20 μl of liposome dispersion in 40 μl of serum-free medium. After an incubation time of about 30 min at room temperature, 20 μl per well of the lipoplex dispersion was pipetted onto the cells.

After a transfection time of 24 h, the medium is changed and the cells are incubated for a further 48 h. Thereafter the medium is removed and the cells are lysed with Triton X-100. The lysate is treated with chlorophenol red β-galactopyranoside (Roche Diagnostics, 1 mg/ml in HBSS) as a substrate for β-galactosidase, and the optical density at different intervals is determined with a microplate reader (Dynex) (measurement wavelength 540 nm, reference wavelength 630 nm).

1. Sulfur-containing amphiphiles of the general formula I: 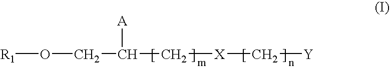
in which
R₁ denotes a straight or branched chain, saturated or unsaturated alkyl or acyl residue with 10-24 carbon atoms,
a denotes a group O—R₂ or CH₂—O—R₂, in which R₂ has the meaning given for R₁ and may be the same as R₁ or different from R₁, where with the presence of a steric center, the methine carbon atom connected to A can be present in R- or S-configuration or racemic, X denotes a group 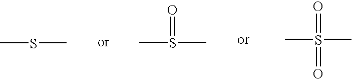
Y denotes a group N⁺R₃R₄R₅Z⁻ or a group NR₃R₄, where R₃-R₅, independently of each other, denote hydrogen, an alkyl group with 1-4 carbon atoms, a group —(CH₂)ᵢ—OH, or a group —(CH₂)ᵢ—NH₂ with i=2-6 and Z⁻ denotes a pharmaceutically acceptable anion, and where m and n, independently of each other, denote an integer 1-6.

2. Compounds according to claim 1, wherein the residue R₁ is an acyl residue from the group of lauroyl, myristoyl, palmitoyl, steroyl, oleoyl, linoloyl, or linoleoyl, m=2 and n=3.

3. Compounds according to claim 1 or 2, wherein R₁ is a lauroyl, myristoyl or oleoyl residue, the group A is a lauroyloxy, myristoyl or oleoyl, m=2 and n=3, X is a —SO— or —SO₂— group, and Y is an —NH₃⁺Z⁻ group or an —NH₂ group.

4. Compounds according to one of claims 1-3, wherein the pharmaceutically acceptable anion is an ion from the group of halide, acetate, trifluoroacetate, mesylate, besylate, phosphate, tartrate, or citrate.

5. Use of a reagent for the transfer of biologically active, anionic macromolecules into eukaryotic cells for pharmaceutical or diagnostic purposes, wherein aggregates are formed from the reagent, which contains at least one compound of claims 1-4, where in addition further lipoid compounds may be admixed in different proportions, with the biologically active, anionic macromolecules, and these aggregates are brought into contact with the cells in vivo or in vitro.

6. Use of a reagent according to claim 5, wherein as the biologically active, anionic macromolecules, DNA, RNA, antisense DNA, antisense RNA, oligonucleotides, ribozymes, peptides or proteins are concerned.

7. Use of a reagent according to claim 5 or 6, wherein the additionally admixed lipoid compounds belong to the phospholipids or steroid classes, 1,2-dioleoyl-sn-glycero-3-phosphoethanolamine being particularly suitable.

8. Use of a reagent according to one of claims 5-7, wherein the reagent is present as a dispersion in aqueous media or as a solution in a solvent miscible with water, and in the case of an aqueous dispersion cryoprotective media from the group of lactose, trehalose, sucrose, glucose, fructose, galactose, maltose, mannitol or polyethylene glycol can be dissolved at the same time.

9. Use of a reagent according to one of claims 5-8, wherein the biologically active anionic macromolecules may be present as complexes with polycationic molecules from the group of spermine, spermidine, protamine sulfate, histone H1, histone H2A, histone H2B, histone H3, histone H4, HMG1 or HMG17 protein.

10. Use of a reagent according to one of claims 5-9, wherein the aggregates formed from the lipids and the anionic macromolecules are stored in lyophilized form, and are rehydrated in a suitable aqueous medium before use.
